(19) United States
(12) Patent Application Publication
(10) Pub. No.: US 2025/0134501 A1
(43) Pub. Date: May 1, 2025

(54) BIOPSY DEVICES AND METHODS

(71) Applicant: Promaxo, Inc., Oakland, CA (US)

(72) Inventors: Akram GHOLAMI PAREH, Oakland, CA (US); Paul BRATHWAITE, Oakland, CA (US); Srirama VENKATARAM, Oakland, CA (US); Aleksandar Nacev, Oakland, CA (US)

(21) Appl. No.: 19/009,518

(22) Filed: Jan. 3, 2025

Related U.S. Application Data
(63) Continuation of application No. PCT/US2023/026949, filed on Jul. 5, 2023.
(60) Provisional application No. 63/367,787, filed on Jul. 6, 2022.

(51) Int. Cl. A61B 10/02

(52) U.S. Cl. CPC A61B 10/0275; A61B 10/0283; A61B 2010/0208

(57) ABSTRACT

Biopsy devices and methods are disclosed. A biopsy device can include a needle assembly and a carrier. The needle assembly can include a cutting cannula and a needle stylet. The needle stylet can include a body forming a longitudinal conduit. The needle stylet can further include a notch in the body, and apertures along the notch. The carrier can include a housing configured to slidably receive the needle stylet, wherein the housing includes a proximal end and a distal end. The carrier further includes an extraction conduit extending from the proximal end toward the distal end, and a storage container fluidically coupled to the extraction conduit and to a vacuum source. The tissue sample is impelled from the notch to the extraction conduit upon actuation of the pump, and the tissue sample is extracted along the extraction conduit to the storage container upon actuation of the vacuum source.

### CROSS-REFERENCE

This application is a continuation of International Patent Application No. PCT/US2023/026949, filed Jul. 5, 2023, which claims priority to U.S. Provisional Application No. 63/367,787, filed Jul. 6, 2022, which is hereby incorporated by reference in its entirety for all purposes.

### BACKGROUND

Biopsy needles and related devices can be used to sample and/or harvest tissue.

### SUMMARY

In one general aspect, the present disclosure provides a biopsy assembly, comprising a needle assembly comprising a cutting cannula and a needle stylet. The needle stylet comprises a body forming a longitudinal conduit, wherein the longitudinal conduit is fluidically coupled to a pump. The needle stylet further comprises a notch in the body, and apertures along the notch. The apertures form transverse openings through the body to the longitudinal conduit. The biopsy assembly further comprises a carrier, comprising a housing configured to slidably receive the needle stylet, wherein the housing comprises a proximal end and a distal end. The carrier further comprises an extraction conduit extending from the proximal end toward the distal end, and a storage container fluidically coupled to the extraction conduit and to a vacuum source. The cutting cannula is movable distally relative to the notch to excise a tissue sample, wherein the cutting cannula is movable proximally to release the tissue sample. The tissue sample is impelled from the notch to the extraction conduit upon actuation of the pump, and the tissue sample is extracted along the extraction conduit to the storage container upon actuation of the vacuum source.

In another aspect, the present disclosure provides a biopsy assembly, comprising a needle assembly, comprising an inner stylet comprising a hollow body forming a longitudinal conduit, a solid distal face, a lateral notch, and through-holes defined through the hollow body along the lateral notch. The needle assembly further comprises a cutting cannula slidably disposed around the inner stylet. The biopsy assembly further comprises a carrier, comprising a housing configured to slidably receive the needle assembly, wherein the housing comprises a proximal end and a distal end. The carrier further comprises an extraction conduit extending from the proximal end toward the distal end, and a storage container fluidically coupled to a vacuum source.

In another aspect, the present disclosure provides a method for performing a biopsy, the method comprising advancing a biopsy needle and a cutting cannula into tissue, wherein the biopsy needle comprises a notch configured to receive a tissue sample, retracting the cutting cannula proximally past the notch to release the tissue sample, and pumping fluid through the biopsy needle into apertures defined along the notch to expel the tissue sample from the notch into an extraction conduit. The method further comprises generating a vacuum in the extraction conduit to withdraw the tissue sample into a storage container fluidically coupled to the extraction conduit.

### BRIEF DESCRIPTION OF THE FIGURES

The novel features of the various aspects are set forth with particularity in the appended claims. The described aspects, however, both as to organization and methods of operation, may be best understood by reference to the following description, taken in conjunction with the accompanying drawings in which:

FIG. 1A is a plan view of a distal portion of a needle stylet, according to various aspects of the present disclosure.

FIG. 1B is an elevation view of the distal portion of the needle stylet of FIG. 1A, according to various aspects of the present disclosure.

FIG. 1C is a cross-sectional, elevation view of the distal portion of the needle stylet of FIG. 1A taken along the plane 1C in FIG. 1A, according to various aspects of the present disclosure.

FIG. 2 is a perspective view of a carrier including a housing, an extraction tube, a motor, and a tissue storage container, wherein the tissue storage container is fluidically coupled to a vacuum tube, according to various aspects of the present disclosure.

FIG. 3 is a perspective view of the carrier of FIG. 2 showing the tissue storage container as transparent for illustrative purposes, according to various aspects of the present disclosure.

FIG. 4 is an elevation view of the carrier of FIG. 2, according to various aspects of the present disclosure.

FIG. 5 is a plan view of a biopsy device including the carrier of FIG. 2 and a needle assembly positioned within the housing of the carrier, wherein the needle assembly includes the needle stylet of FIG. 1 and a cutting cannula, and wherein the needle assembly is in a retracted position relative to the housing, according to various aspects of the present disclosure.

FIG. 6 is a plan view of the biopsy device of FIG. 5, wherein the needle assembly is in an extended position relative to the housing of the carrier, according to various aspects of the present disclosure.

FIG. 7 is an elevation view of a distal portion of the biopsy device of FIG. 5 depicting the needle assembly in an intermediate position between the retracted position of FIG. 5 and the extended position of FIG. 6, according to various aspects of the present disclosure.

FIG. 7A is a detail view of the distal portion of the biopsy device of FIG. 7 with portions shown as transparent for illustrative purposes and further depicting the cutting cannula further retracted relative, according to various aspects of the present disclosure.

The accompanying drawings are not intended to be drawn to scale. Corresponding reference characters indicate corresponding parts throughout the several views. For purposes of clarity, not every component may be labeled in every drawing. The exemplifications set out herein illustrate certain embodiments of the invention, in one form, and such exemplifications are not to be construed as limiting the scope of the invention in any manner.

### DETAILED DESCRIPTION

A biopsy is a procedure that often involves removing a tissue sample from the body and examining it. For example, the tissue sample can be examined for various diseases. One type of biopsy is a needle biopsy, which involves inserting a needle into the body of a patient (e.g. through the skin) to collect tissue from a suspicious area. Needle biopsy techniques include a core needle biopsy (CNB) technique and a vacuum-assisted biopsy (VAB) technique.

CNBs may utilize a thinner needle than VABs and, thus, may be less invasive and/or associated with less bleeding and/or discomfort. For example, the needles for CNBs are typically 18- to 14-gauge needles, whereas the needles for VABs are typically 14- to 7-gauge needles (e.g. having an outer diameter between 2.1 and 4.6 mm diameters). As a result, CNBs may result in fewer surgical complications than VABs in certain instances. For example, larger diameter needles can be associated with a higher risk of bleeding, particularly for VABs relative to CNBs. In the article “Ultrasound-guided diagnostic breast biopsy methodology: a retrospective comparison of the 8-gauge vacuum-assisted biopsy approach versus the spring-loaded 14-gauge core biopsy approach” by S. Povoski, R. Jimenez, and W. Wang in World Journal of Surgical Oncology, 2011; Vol. 9, pp. 87-101, which is incorporated by reference herein in its entirety, the authors found that a VAB with an 8-gauge needle had a higher risk of post-procedural bleeding than a CNB with a 14-gauge needle for benign tumors; however, there was no difference between these techniques for malignant lesions.

VAB devices are used in connection with a vacuum system, which can obtain a larger tissue sample from each insertion. However, some studies show that differences between CNB and VAB techniques is not statistically significant. Although a VAB utilizing, for example, a 14-gauge automated gun, can yield more tissue in a shorter amount of time than a CNB, the results show that the use of the VAB technique is not usually necessary for diagnosis. Using the VAB technique may increase confidence in excluding malignancy in some cases; however, a second biopsy is often recommended regardless of the biopsy method.

VAB devices can include a coaxial gun with the tissue samples returning through the cannula and being recovered outside of the body (e.g. Mammotome® Tru-Core™ II automatic biopsy device) or being stored in a small posterior container (e.g. Atec®, Senorx®, and Mammotome Revolve™ biopsy devices). Coaxial systems can be capable of extracting smaller lesions (e.g. 10 mm vs. 19 mm), and the procedure can be faster.

The mechanisms for creating the vacuum pressure for VABs can be expensive. and high pressure can be required to remove obstructions/debris from the various lumens and ports in these systems.

Aside from sample size, lesion consistency is important in a biopsy procedure. For example, it is often preferable to obtain tissue samples that are neither crushed nor fragmented.

CNB and VAB devices and techniques are further described in the articles “MRI Vacuum-Assisted Breast Biopsies” by R. Plantade and I. Thomassin-Naggara in Diagnostic and Interventional Imaging, September 2014, Vol. 95, Issue 9, pp. 779-801; “Comparison of Automated Versus Vacuum-Assisted Biopsy Methods for Sonographically Guided Core Biopsy of the Breast” by L. Philpotts, R. Hooley, and C. Lee in American Journal of Roentgenology, February 2003, Vol. 180, Issue 2, pp. 347-51; and “Differentiating Vacuum-Assisted Breast Biopsy from Core Needle Biopsy: Is It Necessary?” by S. Nakano, Y. Imawari, A. Mibu, M. Otsuka, and T. Oinuma in The British Journal of Radiology, December 2018, Vol. 91, 20180250, which are incorporated by reference herein in their respective entireties.

VABs can provide certain advantages, such as acquisition of larger and/or more intact tissue samples, faster acquisition time, and/or the ability to store multiple tissue samples in a posterior container. However, existing VAB techniques utilize a larger diameter needle and cannula for excising and extracting the tissue sample, which may be less preferable in certain circumstances in comparison to CNB techniques that utilize a smaller diameter needle and cannula, for example. For example, a larger diameter needle results in a larger incision and may increase the risk of one or more complications in certain instances.

As further described herein, a smaller diameter biopsy needle can be utilized in combination with extraction features that remove the tissue sample to a posterior tissue sample storage volume along a separate extraction conduit. For example, the extraction conduit can be separate and distinct from the biopsy needle and can extend from a distal notch in the biopsy needle to a posterior tissue sample storage volume. In certain instances, the biopsy needle can include features to facilitate expulsion of the excised and harvested tissue from the distal notch in the biopsy needle. For example, the distal notch in the biopsy needle can be coupled to the posterior tissue sample storage volume outside the needle by the extraction conduit. Suction forces can facilitate expulsion of the tissue sample from the notch into the extraction conduit. In such instances, tissue samples are not extracted through the cutting cannula and needle stylet therein, therefore, a thinner needle similar in size to a core needle biopsy needle can be used to excise the tissue sample. As a result, bleeding and/or discomfort from the biopsy needle and associated incision can be reduced. Additionally or alternatively, the accumulation of debris that may clog and/or at least partially obstruct the vacuum ports can be minimized by using a larger diameter extraction conduit than those of conventional VAB cannulas.

In various aspects of the present disclosure, the smaller diameter biopsy needle can be the size of a biopsy needle utilized in CNB procedures (e.g. 14-gauge to 18-gauge) or smaller diameter. For example, a biopsy device in accordance with various aspects of the present disclosure can utilize a core biopsy needle that is coupled to a posterior container having multiple compartments and designed to store multiple tissue specimens. Additionally or alternatively, as further described herein, the core biopsy needle can include features that allow for removal of a tissue sample effectively, efficiently, and in a shorter period of time than existing techniques.

In various aspects of the present disclosure, the biopsy device also includes a biopsy needle carrier, tissue sample storage volume, an extraction conduit comprising a receiving portion and a connecting portion, and a vacuum source, or vacuum pressure-generating device. The biopsy device can be used with both manual biopsy needle devices and automated biopsy needle devices (e.g. an automated biopsy gun). In various instances, the tissue harvesting/incising process with the biopsy devices disclosed herein is the same as the harvesting/incising process of conventional core biopsy needles; however, the extraction technique for removing the tissue sample from the needle to a tissue sample storage volume is different.

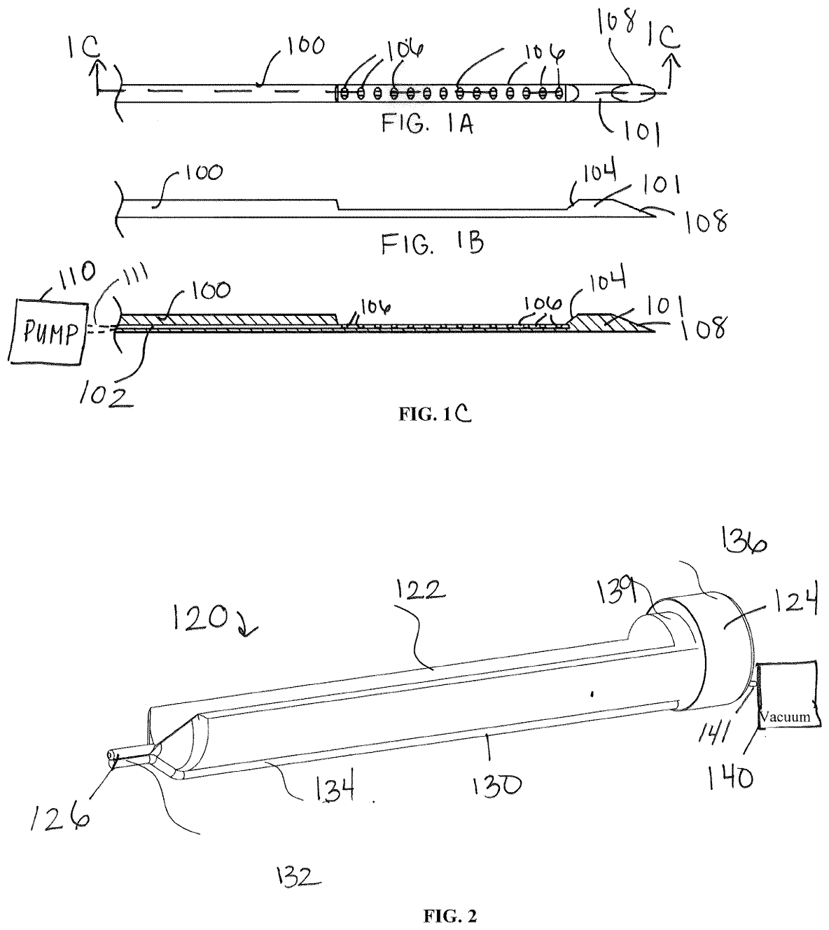

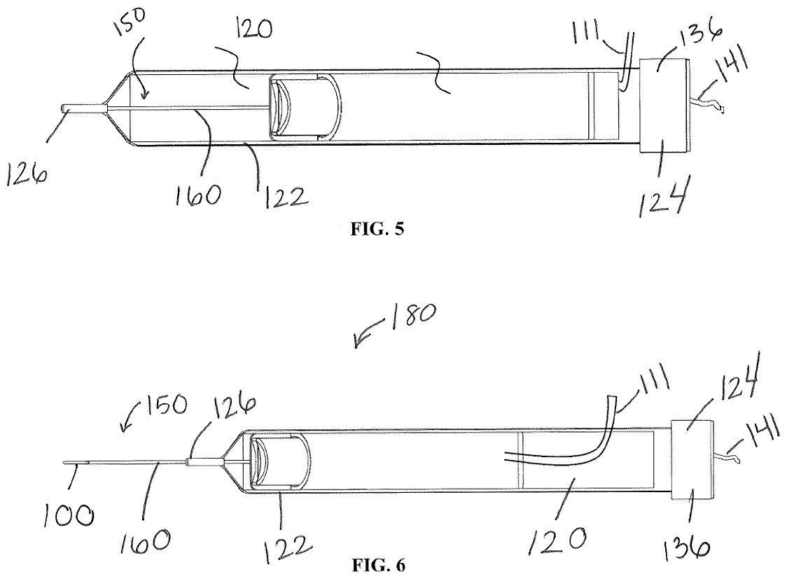

Referring primarily to FIGS. 1A-1C, an inner stylet 100 of a biopsy needle assembly 150 (FIGS. 5-7) is shown. The inner stylet 100 is similar in many aspects to the inner stylet of a needle for CNB techniques. For example, the inner stylet 100 can be dimensioned to fit within a cutting cannula that is 14-gauge or smaller. The inner stylet 100 includes a body 101 defining a central longitudinal conduit 102 therethrough.

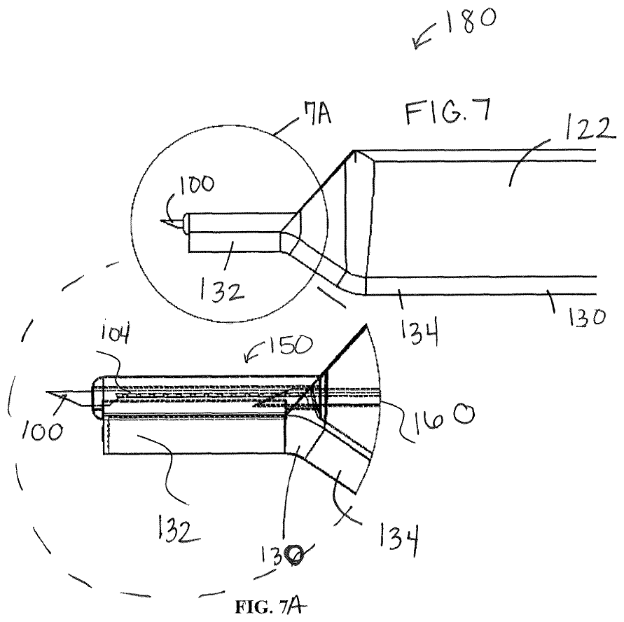

The central longitudinal conduit 102 extends from a proximal end of the inner stylet 100 toward a distal end thereof. The distal end of the inner stylet 100 forms a face or edge 108, which is configured to pierce tissue along with the cutting cannula 160 (see FIG. 7A) positioned therearound. The distal end of the inner stylet 100 is solid. In other words, the edge 108 defines a solid face for penetrating tissue.

Unlike conventional biopsy needles, the inner stylet 100 also includes a distal notch 104 defined in the body 101 and an array of apertures 106 along the distal notch 104. The apertures 106 are arranged in a longitudinal row. Each apertures 106 forms a transverse opening through the body 101 to the central longitudinal conduit 102.

In various aspects, the central longitudinal conduit 102 is fluidically coupled to a pressure-generating device, such as a pump 110, by a fluid line or conduit 111. For example, the pump 110 can be configured to pump fluid through the central longitudinal conduit 102 and into the apertures 106 in the distal notch 104. As further described herein, pushing or pumping fluid through the apertures 106 can facilitate removal of a tissue sample from the inner stylet 100 and into an extraction conduit 130 (FIGS. 2-4) coupled thereto.

In various instances, a biopsy assembly 180 (FIGS. 5-7) includes a biopsy needle assembly 150, which includes the inner stylet 100 and a cutting cannula 160 (FIG. 7A) movably disposed relative to the inner stylet 100. The biopsy assembly 180 can also include a carrier 120 configured to receive the biopsy needle assembly 150, i.e. the cutting cannula 160 and inner stylet 100 therein. Referring primarily to FIGS. 2-4, the carrier 120 includes a housing 122 that is configured to slidably receive the biopsy needle assembly 150. The housing 122 includes a proximal end 124 and a distal end 126. Between the proximal end 124 and the distal end 126 the housing 122 forms a trough that is sized and dimensioned to receive the biopsy needle assembly 150. The distal end 126 defines a tubular passage sized and dimensioned to receive the distal end of the needle assembly therethrough. The biopsy needle assembly 150 is slidably disposed within the distal end 126 and is configured to protrude distally through the tubular passage in various instances, as further described herein. The proximal portion of the needle assembly is configured to rest in the trough formed by the housing 122.

The carrier 120 also includes an extraction conduit 130 extending along the housing 122. A distal portion of the extraction conduit 130 forms a receiving portion 132, which is configured to receive a tissue sample from the needle assembly and, more specifically, from the distal notch 104 in the inner stylet 100. The receiving portion 132 transitions to a connecting portion 134, which extends proximally to a tissue sample storage volume 136. The tissue sample storage volume 136 is fluidically coupled to the extraction conduit 130 and to a vacuum source 140 by a vacuum conduit 141. Upon generation of sufficient vacuum pressure, a tissue sample pushed from the inner stylet 100 into the receiving portion 132 can be drawn proximally along the extraction conduit 130 into the tissue sample storage volume 136.

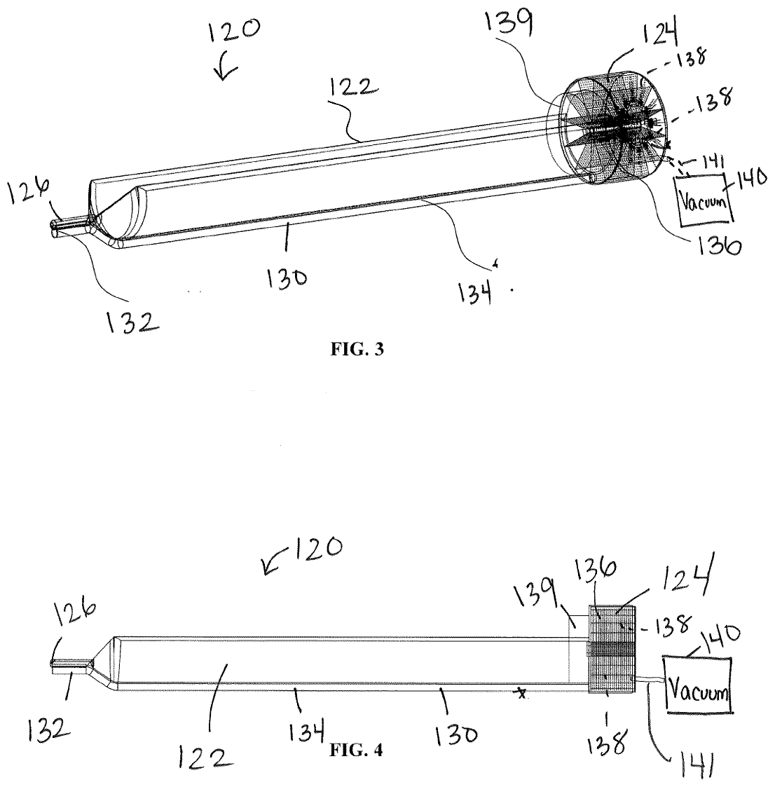

Referring primarily to FIGS. 3 and 4, the tissue sample storage volume 136 can be segmented or partitioned into discrete storage compartments 138 disposed around the perimeter of the tissue sample storage volume 136. The carrier 120 also includes a motor 139, which is operably configured to rotate the tissue sample storage volume 136. In various instances, the tissue sample storage volume 136 is rotatable by the motor 139 relative to the housing 122 and the proximal end of the extraction conduit 130 to fluidically couple one of the storage compartments 138 with the extraction conduit 130. The storage compartments 138 can define discrete, sealed compartments that are sized to receive, hold, and store a tissue sample therein. In various instances, each compartment 138 can house a different tissue sample from a different area within the patient's suspicious tissue or margins thereof.

Referring primarily now to FIGS. 5-7, a biopsy assembly 180 is shown. The biopsy assembly 180 includes a needle assembly that includes the inner stylet 100 and a cutting cannula 160) and also includes the carrier 120. The needle assembly is retained within the housing 122 of the carrier 120 and is movable relative to the housing 122 between a retracted, or home, position (FIG. 5) and an extended position (FIG. 6). In the extended position (FIG. 6), the needle assembly is configured to pierce and excise a tissue sample. For example, the cutting cannula 160 can be movable distally relative to the distal notch 104 in the inner stylet 100 to excise a tissue sample. Thereafter, the cutting cannula 160 is movable proximally to release the excised tissue sample from the distal notch 104. Before releasing the excised tissue sample, the biopsy needle assembly 150 can be retracted from the tissue and returned to the retracted position (FIG. 5). The tissue sample can remain in the biopsy needle assembly 150 until the biopsy needle assembly 150 is returned to the retracted position. In the retracted position, the cutting cannula 160 can be moved coaxially relative to the inner stylet 100 to reveal and expose the distal notch 104.

Upon activation of the pump 110 (FIG. 1C), the tissue sample is impelled or pushed from the distal notch 104 into the receiving portion 132 of the extraction conduit 130. For example, air can be pumped through the apertures 106 from the pump 110 (FIG. 1C) to create an air pump in the distal notch 104 and push the tissue sample into the receiving portion 132.

Upon activation of the vacuum source 140 (FIGS. 2-4), the tissue sample is drawn proximally along the connecting portion 134 of the extraction conduit 130 to the tissue sample storage volume 136. In various instances, the tissue sample storage volume 136 can be fluidically connected to the connecting portion 134 of the extraction conduit 130 on one end and to the vacuum source 140 (FIGS. 2-4) on the opposite end.

Each tissue sample is stored in a compartment 138 of the tissue sample storage volume 136. After the tissue is stored in one of the storage sections, the motor 139 is configured to rotate the tissue sample storage volume 136 so that an empty compartment 138 faces the vacuum source 140 and the connecting portion 132. The vacuum source 140 and other control and supply devices can be integrated into the housing 122 of the carrier 120 and a connection element from the tissue sample storage volume 136 to the vacuum source 140 can be directly integrated into the housing 122.

In various instances, one or more sterile components are used for each patient. In some instances, certain components of the biopsy assembly 180 can be sterilized components and other components can merely be disinfected between uses. For example, a sterile biopsy needle assembly 150 can be used for each patient. In certain instances, the carrier 120 can also be a sterile component that is used for each patient. It can be advantageous to separate the sterile parts from the other parts are merely disinfected and firmly connected to the system. For example, the biopsy needle assembly 150 can be an independent component that is easily inserted and removed from the biopsy assembly 180. Additionally, the carrier 120 can be an independent component that is easily inserted and removed from the biopsy assembly 180. In various instances, the needle assembly, carrier, extraction conduit, and drive elements, including to connection element(s) therefor, can be easily inserted and removable sterile components.

Various aspects of the subject matter described herein are set out in the following numbered examples.

Example 1—A biopsy assembly, comprising a needle assembly comprising a cutting cannula and a needle stylet. The needle stylet comprises a body forming a longitudinal conduit, wherein the longitudinal conduit is fluidically coupled to a pump. The needle stylet further comprises a notch in the body, and apertures along the notch. The apertures form transverse openings through the body to the longitudinal conduit. The biopsy assembly further comprises a carrier, comprising a housing configured to slidably receive the needle stylet, wherein the housing comprises a proximal end and a distal end. The carrier further comprises an extraction conduit extending from the proximal end toward the distal end, and a storage container fluidically coupled to the extraction conduit and to a vacuum source. The cutting cannula is movable distally relative to the notch to excise a tissue sample, wherein the cutting cannula is movable proximally to release the tissue sample. The tissue sample is impelled from the notch to the extraction conduit upon actuation of the pump, and the tissue sample is extracted along the extraction conduit to the storage container upon actuation of the vacuum source.

Example 2—the biopsy assembly of Example 1, wherein the needle stylet comprises a first diameter, wherein the extraction conduit comprises a second diameter, and wherein the second diameter is larger than the first diameter.

Example 3—the biopsy assembly of Examples 1 or 2, wherein the needle stylet is between 14-gauge and 18-gauge.

Example 4—the biopsy assembly of Examples 1, 2, or 3, wherein the extraction conduit is larger than 14-gauge.

Example 5—the biopsy assembly of Examples 1, 2, 3, or 4, wherein the carrier further comprises a motor operably configured to rotate the storage container.

Example 6—the biopsy assembly of Examples 1, 2, 3, 4, or 5, wherein the storage container comprises a plurality of discrete compartments, and wherein the storage container is rotatable relative to the proximal end of the extraction conduit to fluidically couple one of the discrete compartments with the extraction conduit.

Example 7—the biopsy assembly of Examples 1, 2, 3, 4, 5, or 6, further comprising the pump operably configured to selectively pump fluid along the longitudinal conduit through the needle stylet to the apertures.

Example 8—the biopsy assembly of Examples, 1, 2, 3, 4, 5, 6, or 7, wherein the needle stylet comprises a solid distal face.

Example 9. The biopsy assembly of Examples 1, 2, 3, 4, 5, 6, 7, or 8, further comprising the vacuum source operably configured to selectively generate a vacuum force in the extraction conduit.

Example 10—A biopsy assembly, comprising a needle assembly, comprising an inner stylet comprising a hollow body forming a longitudinal conduit, a solid distal face, a lateral notch, and through-holes defined through the hollow body along the lateral notch. The needle assembly further comprises a cutting cannula slidably disposed around the inner stylet. The biopsy assembly further comprises a carrier, comprising a housing configured to slidably receive the needle assembly, wherein the housing comprises a proximal end and a distal end. The carrier further comprises an extraction conduit extending from the proximal end toward the distal end, and a storage container fluidically coupled to a vacuum source.

Example 11—A method for performing a biopsy, the method comprising advancing a biopsy needle and a cutting cannula into tissue, wherein the biopsy needle comprises a notch configured to receive a tissue sample, retracting the cutting cannula proximally past the notch to release the tissue sample, and pumping fluid through the biopsy needle into apertures defined along the notch to expel the tissue sample from the notch into an extraction conduit. The method further comprises generating a vacuum in the extraction conduit to withdraw the tissue sample into a storage container fluidically coupled to the extraction conduit.

In various instances, the biopsy devices and methods disclosed herein can be used in connection with magnetic resonance imaging (MRI) techniques. For example, the biopsy assembly can extend into a field of view being imaged by an MRI device to facilitate placement of the biopsy needle relative to patient's tissue. In certain instances, the biopsy assembly and/or a robotic arm supporting the biopsy assembly can extend through a central bore in the MRI device. Exemplary systems and components are further described in International Application No. PCT/US2021/014628, titled MRI-GUIDED ROBOTIC SYSTEMS AND METHODS FOR BIOPSY, published Jul. 29, 2021, which is incorporated by reference herein in its entirety.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications. variations, changes, substitutions, modifications, and equivalents.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”): the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to.” or other past-tense adjectives are generally not intended to exclude such variants, unless context dictates otherwise.

It is worthy to note that any reference to “one aspect.” “an aspect.” “an exemplification,” “one exemplification,” and the like means that a particular feature, structure, or characteristic described in connection with the aspect is included in at least one aspect. Thus, appearances of the phrases “in one aspect.” “in an aspect.” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A biopsy assembly, comprising:
a needle assembly comprising a cutting cannula and a needle stylet, wherein the needle stylet comprises:
a body forming a longitudinal conduit, wherein the longitudinal conduit is fluidically coupled to a pump;
a notch in the body; and
apertures along the notch, wherein the apertures form transverse openings through the body to the longitudinal conduit; and
a carrier, comprising:
a housing configured to slidably receive the needle stylet, wherein the housing comprises a proximal end and a distal end;
an extraction conduit extending from the proximal end toward the distal end; and
a storage container fluidically coupled to the extraction conduit and to a vacuum source;
wherein the cutting cannula is movable distally relative to the notch to excise a tissue sample, wherein the cutting cannula is movable proximally to release the tissue sample, wherein the tissue sample is impelled from the notch to the extraction conduit upon actuation of the pump, and wherein the tissue sample is extracted along the extraction conduit to the storage container upon actuation of the vacuum source.

2. The biopsy assembly of claim 1, wherein the needle stylet comprises a first diameter, wherein the extraction conduit comprises a second diameter, and wherein the second diameter is larger than the first diameter.

3. The biopsy assembly of claim 1, wherein the needle stylet is between 14-gauge and 18-gauge.

4. The biopsy assembly of claim 3, wherein the extraction conduit is larger than 14-gauge.

5. The biopsy assembly of claim 1, wherein the carrier further comprises a motor operably configured to rotate the storage container.

6. The biopsy assembly of claim 5, wherein the storage container comprises a plurality of discrete compartments, and wherein the storage container is rotatable relative to the proximal end of the extraction conduit to fluidically couple one of the discrete compartments with the extraction conduit.

7. The biopsy assembly of claim 1, further comprising the pump operably configured to selectively pump fluid along the longitudinal conduit through the needle stylet to the apertures.

8. The biopsy assembly of claim 6, wherein the needle stylet comprises a solid distal face.

9. The biopsy assembly of claim 1, further comprising the vacuum source operably configured to selectively generate a vacuum force in the extraction conduit.

10. A biopsy assembly, comprising:
a needle assembly, comprising:
an inner stylet comprising a hollow body forming a longitudinal conduit, a solid distal face, a lateral notch, and through-holes defined through the hollow body along the lateral notch; and
a cutting cannula slidably disposed around the inner stylet; and
a carrier, comprising:
a housing configured to slidably receive the needle assembly, wherein the housing comprises a proximal end and a distal end;
an extraction conduit extending from the proximal end toward the distal end; and
a storage container fluidically coupled to a vacuum source.

11. A method for performing a biopsy, the method comprising
advancing a biopsy needle and a cutting cannula into tissue, wherein the biopsy needle comprises a notch configured to receive a tissue sample;
retracting the cutting cannula proximally past the notch to release the tissue sample; pumping fluid through the biopsy needle into apertures defined along the notch to expel the tissue sample from the notch into an extraction conduit; and generating a vacuum in the extraction conduit to withdraw the tissue sample into a storage container fluidically coupled to the extraction conduit.
